(19) United States
(12) Patent Application Publication
(10) Pub. No.: US 2021/0396655 A1
(43) Pub. Date: Dec. 23, 2021

(54) Method and Device for Analyzing Biological Material

(71) Applicant: Robert Bosch GmbH, Stuttgart (DE)

(72) Inventor: Tino Frank, Luzern (CH)

(21) Appl. No.: 17/292,799

(22) PCT Filed: Nov. 8, 2019
(86) PCT No.: PCT/EP2019/080713

§ 371 Date: May 11, 2021

(30) Foreign Application Priority Data
Nov. 15, 2018 (DE) 10 2018 219 483.1

(51) Int. Cl. G01N 21/27; C12Q 1/686

(52) U.S. Cl. CPC G01N 21/272; G01N 2201/129; G01N 2201/121; C12Q 1/686

(57) ABSTRACT

A method for analyzing biological material includes reading in a measurement signal, a first reference signal and a second reference signal. The method further includes determining noise in the measurement signal in order to produce noise data, applying the noise data to the first reference signal and to the second reference signal in order to generate an adjusted first reference signal and an adjusted second reference signal, and transforming the measurement signal, the adjusted first reference signal, and the adjusted second reference signal into a frequency distribution form in order to produce a measurement signal distribution, a first reference distribution and a second reference distribution. Additionally the method includes performing a cluster analysis using the measurement signal distribution, the first reference distribution, and the second reference distribution to determine, in accordance with a result of the cluster analysis, whether the biological material has the first property or the second property.

### PRIOR ART

The invention proceeds from a device or a method according to the preamble of the independent claims. The subject matter of the present invention is also a computer program.

One challenge in microfluidic systems can be, for example, avoiding or monitoring air bubbles. In particular upon application of temperatures close to the boiling point, such air bubbles can increasingly occur. For example, a polymerase chain reaction can require process steps in which temperatures in this range are used. Since an analysis or evaluation typically takes place here via optical signals, such air bubbles can represent an interference source, which can in particular cause noise of measured intensity values.

### SUMMARY OF THE INVENTION

Against this background, the approach presented here presents a method, furthermore a device which uses this method, and finally a corresponding computer program according to the main claims. Advantageous refinements and improvements of the device specified in the independent claim are possible by way of the measures set forth in the dependent claims.

According to embodiments, in particular an amplification decision for a noisy, microfluidic or optofluidic analysis of biological material can be enabled in a reliable manner. In particular, an evaluation of optofluidic data can be carried out here in spite of noise caused by gas bubbles. Such an analysis can be, for example, a so-called quantitative real-time PCR (qPCR; PCR=polymerase chain reaction). In particular a presence of ultrasmall quantities of a specific DNA section (DNA=deoxyribonucleic acid) can be detected both quantitatively and also qualitatively. Therefore, according to embodiments, in particular a process for the amplification decision for noisy microfluidic qPCR analyses can be provided. In the amplification decision, it can be decided whether or not measurement data indicate an amplification of molecules to be detected by the analysis.

Microfluidic systems can enable an analysis of small sample quantities with a high sensitivity. Automation, miniaturization, and parallelization can moreover enable a reduction of manual steps, and an alleviation of errors thus caused, in microfluidic systems. Microfluidic systems which can execute fluidic and analytic processes via an optical system are associated in particular with optofluidics. Optofluidic processes can use properties of light to generate results. During the analysis, noise resulting due to gas bubbles or air bubbles can also be taken into consideration and calculated out without complex and impractical improvement of signal amplitude or probe design. It is thus possible to prevent, for example, that an evaluation of such noisy measurement data can be simplified and a reduction of specificity and sensitivity of the analysis caused by such bubbles can be prevented. To maintain these important variables of the analysis and thus of the underlying diagnostic system in spite of bubble interference, in particular signal processing methods can advantageously be used.

According to embodiments, in particular an unbiased decision process with respect to a result of the analysis can advantageously be achieved. Thus, for example, a curve fitting on two models to be expected—these are a linear function or a sigmoid function in qPCR—can be omitted. In contrast to curve fitting, in which the smaller error (goodness of fit) was assumed as the existing model, i.e., would represent the decision as to whether the behavior of the data corresponds to a linear function or a sigmoid function, according to embodiments, a statistically relevant difference can be obtained in particular even at high noise level, at which residues (errors of the curve fitting) would be too close to one another. Moreover, required test data can be reduced to define and validate acceptance criteria. Test data can also be omitted by measuring the noise and generating noise-analogous, generic model data. After a model decision, a fitting can be carried out, which enables a simple adaptation of the curve fitting principle. In spite of bubble formation during an analysis, in particular a qPCR, in a microfluidic system or in a microfluidic device, a reliable amplification difference can be made with a minimal use of control reactions. The analysis can run fully automatically and manages without user input. No additional, analysis-specific parameters, except for the measurement data themselves, have to be input for the evaluation. Due to a universality of the presented methods, they are particularly suitable in qPCR systems at the usage location or point of care. Usage errors due to incorrect parameter selection can be minimized or prevented.

A method for analyzing biological material is presented, wherein the method includes the following steps: reading a measurement signal, a first reference signal, and a second reference signal, wherein the measurement signal represents acquired optofluidic data of the biological material, wherein the first reference signal represents first optofluidic model data that correspond to a first property of the biological material, wherein the second reference signal represents second optofluidic model data that correspond to a second property of the biological material;

ascertaining a noise of the measurement signal to generate noise data;

applying the noise data to the first reference signal and to the second reference signal to generate an adapted first reference signal and an adapted second reference signal;

transforming the measurement signal, the adapted first reference signal, and the adapted second reference signal into a frequency distribution form to generate a measurement signal distribution, a first reference distribution, and a second reference distribution; and

performing a cluster analysis using measurement signal distribution, the first reference distribution, and the second reference distribution to establish as a function of a result of the cluster analysis whether the biological material has the first property or the second property.

The biological material can be a liquid comprising target molecules, for example DNA sections. The method can be executed using a microfluidic system or a microfluidic device. The measurement signal can represent a signal acquired using an optical sensor. A characteristic of the measurement signal can be dependent on a property of the biological material. A property of the biological material can be determined, for example, by a type and/or concentration of target molecules in the biological material. A reference signal can be a stored signal. The optofluidic model data of a reference signal can, for example, have been acquired previously in a reference measurement or can have been previously simulated or calculated. The first reference signal can correspond to a first type or quantity of a target molecule. The second reference signal can correspond to a second type or quantity of a target molecule. The first reference signal can also correspond to a presence of a target molecule and the second reference signal can correspond to a non-presence of the target molecule, wherein a presence can be defined, for example, by a concentration of the target molecule in the biological material lying above a threshold value. In the step of ascertaining, the noise data can be generated, for example, by a suitable filtering of the measurement signal. In the step of applying, the noise data and the reference signals can be combined. For example, the reference signals can be overlaid with the noise data. The adapted reference signals can have a noise characteristic corresponding to the measurement signal. In the step of performing, an analysis signal can be determined which can depict the result of the cluster analysis and can thus indicate the property of the biological material determined using the measurement signal and the reference signals.

One embodiment of the method can enable a decision process based on a cluster analysis or a cluster method. In this case, a measured signal can be compared to two generic, result-typical reference signals. To simplify the cluster analysis, the noise of the measurement data can be measured and reference data having the same noise can be generated. The transformation of the signals into a distribution form enables a more robust decision with respect to a result of the analysis.

According to one embodiment, the method includes a step of acquiring the optofluidic data of the biological material to provide the measurement signal. The step of acquiring can be executed by means of microfluidic apparatuses. Such an embodiment offers the advantage that small quantities of biological material can also be analyzed with high sensitivity in an automatic and miniaturized manner.

In the step of reading, a measurement signal can also be read which represents optofluidic data of the biological material acquired by means of a quantitative and additionally or alternatively qualitative polymerase chain reaction. In this case, in the step of reading, a first reference signal that has a sigmoid curve and a second reference signal that has a linear curve can be read. The first property of the biological material can result in an amplification of at least one target molecule of the biological material by the analysis. In this case, the second property of the biological material can result in an absence of an amplification of the at least one target molecule by the analysis. In the step of transforming, the adapted first reference signal and the adapted second reference signal can be transformed in such a way that the first reference distribution is a bimodal distribution and the second reference distribution is a unimodal distribution. Such an embodiment offers the advantage that an evaluation or amplification decision for a polymerase chain reaction can be carried out in a noncomplex, reliable, and accurate manner.

Furthermore, in the step of performing, the cluster analysis can be performed by means of a k-means algorithm having a predefined distance measure. The first reference distribution can represent a first cluster here. The second reference distribution can represent a second cluster. The result of the cluster analysis can indicate whether, in consideration of the distance measure, the measurement signal distribution falls in the first cluster or in the second cluster. Instead of the k-means algorithm, another algorithm from the field of cluster analysis or another suitable field can also be used. Such an embodiment offers the advantage that a reliable amplification decision for the analysis on the biological material can be made in a simple manner.

Moreover, in the step of ascertaining, the noise of the measurement signal can be ascertained over a course of the analysis and additionally or alternatively by means of a sliding window process and additionally or alternatively using a noise measure to generate a functional relationship between the noise and the course of the analysis as the noise data. The noise measure can be a local standard deviation, a local signal-to-noise ratio, or the like. The course of the analysis can represent a passage of the time and additionally or alternatively a cycle number of the analysis. Such an embodiment offers the advantage that a decision as to whether the measurement signal corresponds more to the first reference signal or the second reference signal can be made even more robustly and reliably.

In the step of applying, random numbers or pseudorandom numbers dependent on the noise data can be added to the model data of the reference signals. Such an embodiment offers the advantage that the adapted reference signals can be obtained in a simple manner to accurately image the noise present in the measurement signal.

The method can furthermore include a step of scaling the read measurement signal by projecting absolute values on a predefined value interval. In this case, in the step of ascertaining, the noise of the scaled measurement signal can be ascertained. Such an embodiment offers the advantage that the cluster analysis can be facilitated or a more exact result of the cluster analysis can be enabled.

This method can be implemented, for example, in software or hardware or in a mixed form of software and hardware, for example in a control unit.

The approach presented here furthermore provides a device which is designed to carry out, activate, or implement the steps of a variant of a method presented here in corresponding apparatuses. The object underlying the invention can also be achieved rapidly and efficiently by this embodiment variant of the invention in the form of a device.

For this purpose, the device can include at least one processing unit for processing signals or data, at least one storage unit for storing signals or data, at least one interface to a sensor or an actuator for reading sensor signals from the sensor or for outputting data or control signals to the actuator, and/or at least one communication interface for reading or outputting data, which are embedded in a communication protocol. The processing unit can be, for example, a signal processor, a microcontroller, or the like, wherein the storage unit can be a flash memory, an EEPROM, or a magnetic storage unit. The communication interface can be designed to read or output data in a wireless and/or wired manner, wherein a communication interface which can read or output data in a wired manner can read these data, for example, electrically or optically from a corresponding data transmission line or can output these data in a corresponding data transmission line.

A device can be understood in the present case as an electrical device which processes sensor signals and outputs control and/or data signals as a function thereof. The device can include an interface which can be designed as hardware and/or software. In a hardware design, the interfaces can be part of a so-called system ASIC, for example, which includes greatly varying functions of the device. However, it is also possible that the interfaces are separate integrated circuits or at least partially consist of discrete components. In a software design, the interfaces can be software modules which are present on a microcontroller in addition to other software modules, for example.

The device can be embodied as a microfluidic device, in particular as a so-called chip laboratory or LoC (lab-on-chip). Alternatively, the device can be embodied as a control unit inside or outside a microfluidic device. In one advantageous embodiment, a control of a microfluidic device is carried out by the device. The device can also be designed to guide an evaluation of measurement data acquired by means of microfluidic apparatuses. For this purpose, the device can access sensor signals such as the measurement signal, for example. The device can be designed to provide an output signal which represents a result or intermediate result of the analysis. The result or intermediate result can be a statement as to whether the biological material has the first property or the second property.

A computer program product or computer program is also advantageous, having program code, which can be stored on a machine-readable carrier or storage medium such as a semiconductor memory, a hard drive memory, or an optical memory and is used to carry out, implement, and/or activate the steps of the method according to one of the above-described embodiments, in particular when the program product or program is executed on a computer or a device.

Exemplary embodiments of the approach presented here are illustrated in the drawings and explained in greater detail in the following description. In the figures:

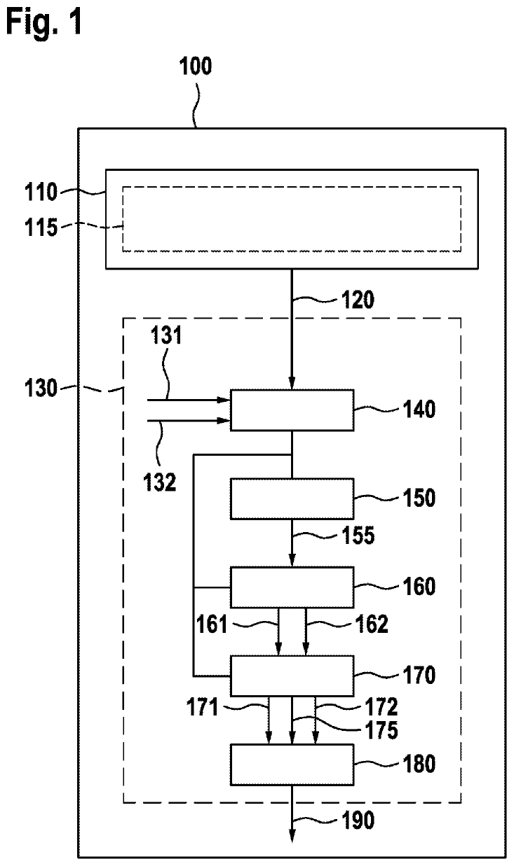

FIG. 1 shows a schematic illustration of a device according to one exemplary embodiment;

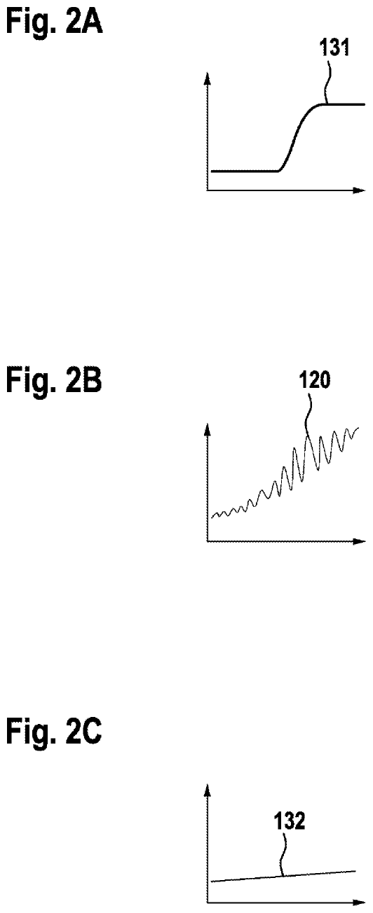

FIG. 2A to FIG. 2C show schematic signal diagrams according to one exemplary embodiment;

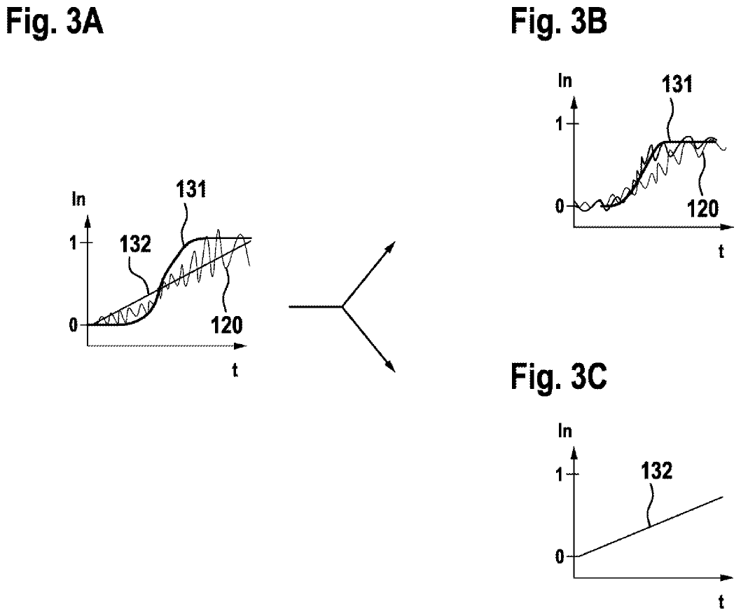

FIG. 3A to FIG. 3C show schematic signal diagrams according to one exemplary embodiment;

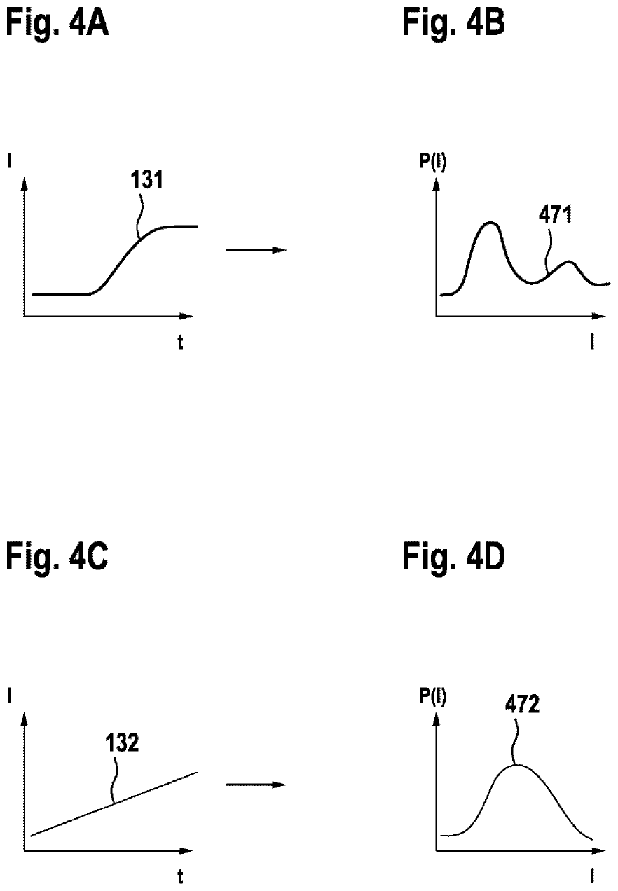

FIG. 4A to FIG. 4D show schematic signal diagrams according to one exemplary embodiment;

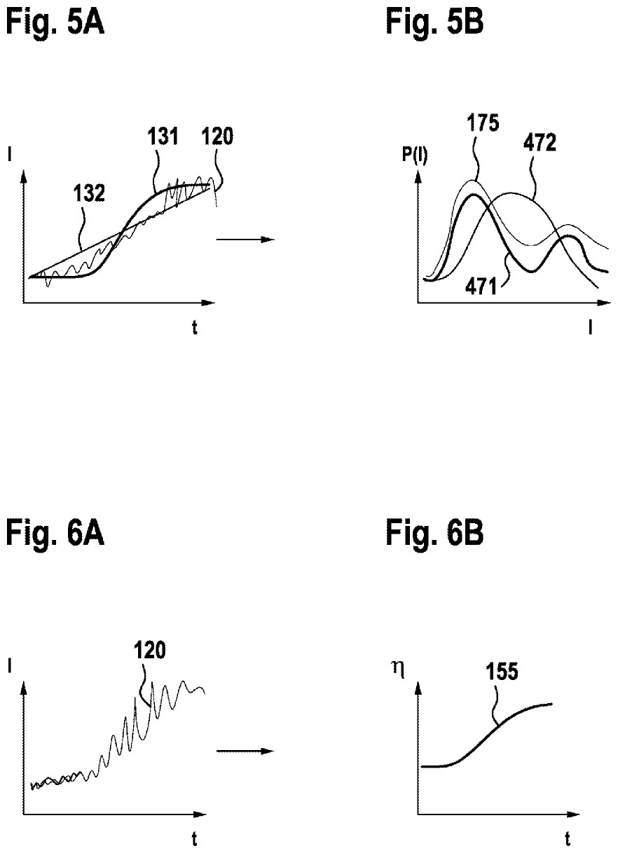

FIG. 5A and FIG. 5B show schematic signal diagrams according to one exemplary embodiment;

FIG. 6A and FIG. 6B show schematic signal diagrams according to one exemplary embodiment;

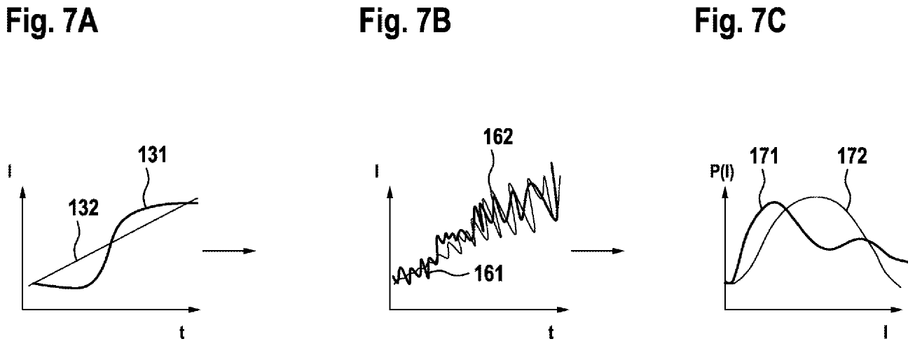

FIG. 7A to FIG. 7C show schematic signal diagrams according to one exemplary embodiment;

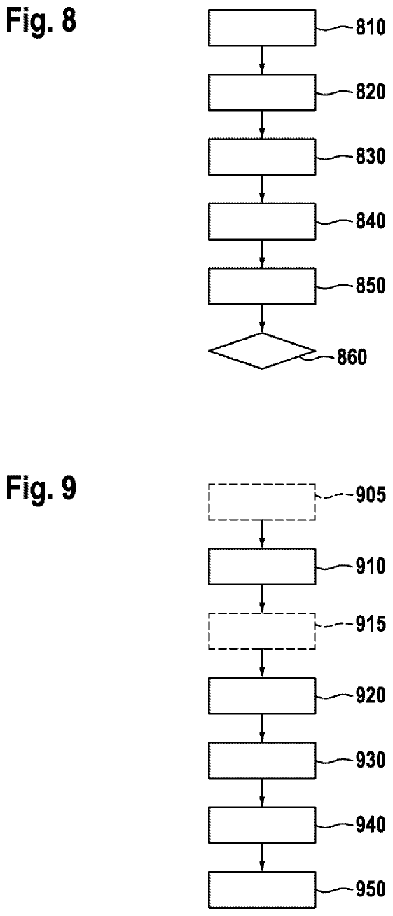

FIG. 8 shows a flow chart of an evaluation process according to one exemplary embodiment; and

FIG. 9 shows a flow chart of a method according to one exemplary embodiment.

In the following description of advantageous exemplary embodiments of the present invention, identical or similar reference signs are used for elements shown in the various figures and acting similarly, wherein a repeated description of these elements is omitted.

FIG. 1 shows a schematic illustration of a device 100 according to one exemplary embodiment. The device 100 is designed to execute an analysis of biological material. The device 100 can also be referred to as an analysis device 100. The biological material includes, for example, genetic material. The device 100 is embodied in particular as a chip laboratory. The device 100 is designed here, more precisely, to execute a quantitative real-time PCR (qPCR; PCR=polymerase chain reaction). The qPCR is executed here in a plurality of cycles. For example, a presence of ultrasmall quantities of a specific DNA section (DNA=deoxyribonucleic acid) is detected quantitatively and/or qualitatively.

The device 100 includes at least one microfluidic apparatus 110. The biological material is introducible, for example, in a cartridge 115 into the device 100 or microfluidic apparatus 110. The cartridge 115 is receivable in the device 100 or the microfluidic apparatus 110. The at least one microfluidic apparatus 110 is designed to acquire optofluidic data of the biological material. For this purpose, the microfluidic apparatus 110 is designed, for example, to acquire light reflected and/or emitted from the biological material.

Furthermore, the at least one microfluidic apparatus 110 is designed to provide a measurement signal 120, for example, using the light originating from the biological material. The measurement signal 120 represents, according to one exemplary embodiment, acquired optofluidic data of the biological material. To excite the biological material, the microfluidic apparatus 110 is designed according to one exemplary embodiment to irradiate the biological material with electromagnetic radiation, for example light.

The device 100 moreover includes a control unit 130, which is also referred to as a control apparatus 130 or control device 130 for analyzing the biological material. The control unit 130 is connected to the at least one microfluidic apparatus 110 in a manner capable of signal transmission. The control unit 130 includes, according to the exemplary embodiment shown here, a read apparatus 140, an ascertainment apparatus 150, an application apparatus 160, a transformation apparatus 170, and a performance apparatus 180.

The read apparatus 140 is designed to read the measurement signal 120. Furthermore, the read apparatus 140 is designed to read a first reference signal 131 and a second reference signal 132. The first reference signal 131 represents first optofluidic model data, which correspond to a first property of the biological material, and the second reference signal 132 represents second optofluidic model data, which correspond to a second property of the biological material. The read apparatus 140 is connected in a manner capable of signal transmission to the ascertainment apparatus 150, to the application apparatus 160, and to the transformation apparatus 170.

The ascertainment apparatus 150 is designed to ascertain a noise of the measurement signal 120. Furthermore, the ascertainment apparatus 150 is designed to generate noise data 155, which represent the ascertained noise of the measurement signal 120. The ascertainment apparatus 150 is connected in a manner capable of signal transmission to the application apparatus 160.

The application apparatus 160 is designed to apply the ascertained noise data 155 to the first reference signal 131 and to the second reference signal 132 to generate an adapted first reference signal 161 and an adapted second reference signal 162. The application apparatus 160 is connected in a manner capable of signal transmission to the transformation apparatus 170.

The transformation apparatus 170 is designed to transform the measurement signal 120, the adapted first reference signal 161, and the adapted second reference signal 162 into a frequency distribution form to generate a measurement signal distribution 175, a first reference distribution 171, and a second reference distribution 172. The transformation apparatus 170 is connected in a manner capable of signal transmission to the performance apparatus 180.

The performance apparatus 180 is designed to perform a cluster analysis using the measurement signal distribution 175, the first reference distribution 171, and the second reference distribution 172. More precisely, the performance apparatus 180 is designed to perform the cluster analysis to establish as a function of a result of the cluster analysis whether the biological material has the first property or the second property. The result of the cluster analysis represents an association of the measurement signal distribution 175 with the first reference distribution 171 or the second reference distribution 172.

The control unit 130 is furthermore designed to output or provide an analysis signal 190. The analysis signal 190 represents a result of the analysis. For example, the analysis signal comprises an item of information about the property of the biological material.

According to one exemplary embodiment, the control unit 130 is also designed to scale the read measurement signal 120, in particular by projecting absolute values of the measurement signal 120 on a predefined value interval. The scaling is optionally executable by means of the read apparatus 140 or the ascertainment apparatus 150. In this case, the ascertainment apparatus 150 is designed to ascertain the noise on the basis of the scaled measurement signal.

In particular, the processes executed by the control unit 130 are also further clarified with reference to the following figures.

FIG. 2A, FIG. 2B, and FIG. 2C show schematic signal diagrams according to one exemplary embodiment. FIG. 2A shows the first reference signal 131 from FIG. 1. The first reference signal 131 has a sigmoid curve or sigma curve. FIG. 2B shows the measurement signal 120 from FIG. 1. The measurement signal 120 is subject to noise. FIG. 2C shows the second reference signal 132 from FIG. 1. The second reference signal 132 has a linear curve.

In other words, FIG. 2A, FIG. 2B, and FIG. 2C show a decision problem of a qPCR curve evaluation. If a qPCR curve was recorded in the form of the measurement signal 120 or in the form of raw data, this was assessed as to whether it is an amplification or not an amplification. The first reference signal 131 represents the amplification. The second reference signal 132 represents no amplification or an absence of amplification. The first reference signal 131 can be described as a sigmoid function having five parameters, as nonlinear, and having S shape. The second reference signal 132 can be described as a linear function having two parameters. The measurement signal 120 is typically based on n measurement points, wherein n corresponds to a number of PCR cycles of the analysis. Because of the system, these measurement points include a noise η. The decision problem then exists as to whether the measurement signal 120 having the noise η corresponds or has similarity more to the first reference signal 131 or to the second reference signal 132.

FIG. 3A, FIG. 3B, and FIG. 3C show schematic signal diagrams according to one exemplary embodiment. The schematic signal diagrams are shown in the form of intensity-time diagrams or intensity-cycle diagrams. The time t or a cycle number is plotted in each case here on the abscissa axes. An intensity In or signal intensity In is plotted in each case on the ordinate axes. FIG. 3A shows the measurement signal 120 from FIG. 2B, the first reference signal 131 from FIG. 2A, and the second reference signal 132 from FIG. 2C in superimposed form. FIG. 3B shows the measurement signal 120 and the first reference signal 131 in superimposed form. FIG. 3C shows the second reference signal 132. In this case, in FIG. 3A, FIG. 3B, and FIG. 3C, the measurement signal 120, the first reference signal 131, and the second reference signal 132 are scaled with respect to the intensity In, for example to an interval between 0 and 1.

More precisely and in other words, FIG. 3A, FIG. 3B, and FIG. 3C show an approach for solving the decision problem mentioned in conjunction with FIG. 2A, FIG. 2B, and FIG. 2C. Instead of a line fitting of the measurement signal 120 against the first reference signal 131 and the second reference signal 132 and an estimation of the error (goodness of fit or Kolmogorov-Smirnov test), which in the case of a large amount of noise η can be amplified in microfluidic devices and systems by air bubbles, a cluster analysis or a cluster method (k-means clustering) using the k-means algorithm is made use of here to maintain a robustness of the analysis of the biological material. For this purpose, the measurement signal 120 is scaled, i.e., absolute values are projected onto an interval [0, 1], and compared to two generic functions, which correspond to the first reference signal 131 and the second reference signal 132. The comparison here is a so-called k-means clustering using a predefined distance measure, for example, the Euler distance, Manhattan distance, or the like, having a total of two clusters, k=2. The generic functions (also called dummy functions) each form a cluster category or a cluster and the measurement signal 120 falls due to the respective smaller distance into one of these clusters. It is possible that the measurement signal 120 forms a separate cluster and the clusters based on the reference signals 131 and 132 coincide. Based on the system and theory, an invalid measurement would exist here, since the measurement signal 120 has a shape not to be expected. This case can be used as a quality control.

FIG. 4A, FIG. 4B, FIG. 4C, and FIG. 4D show schematic signal diagrams according to one exemplary embodiment. The schematic signal diagrams in FIG. 4A and FIG. 4C are shown in the form of intensity-time diagrams or intensity-cycle diagrams. In this case, the time t or a cycle number is plotted in each case on the abscissa axes. An intensity I or signal intensity I is plotted in each case on the ordinate axes. The schematic signal diagrams in FIG. 4B and FIG. 4D are shown in the form of intensity distribution-intensity diagrams. In this case, the intensity I or signal intensity I is plotted in each case on the abscissa axes. An intensity distribution P(I) is plotted in each case on the ordinate axes. FIG. 4A shows the first reference signal 131 from one of the above-mentioned figures. FIG. 4B shows a first reference distribution 471, which is obtained by transforming the first reference signal 131 into a distribution form. FIG. 4C shows the second reference signal 132 from one of the above-mentioned figures. FIG. 4D shows a second reference distribution 472, which is obtained by transforming the second reference signal 132 into a distribution form.

In other words, FIG. 4A, FIG. 4B, FIG. 4C, and FIG. 4D show a first step for the robust cluster decision. Instead of assessing the qPCR or the measurement signal originating therefrom in the form of a cycle-intensity function, a histogram of intensity values is created. For this purpose, a kernel density estimation is performed over the measurement signal or the PCR curve. Since a qPCR curve, amplifying or non-amplifying, is monotonously rising (with noise in the trend monotonously rising), low intensities correspond to early cycles and high intensities to late values. If the first reference signal 131 is now transformed, a bimodal distribution results as the first reference distribution 471. This is explainable by the two plateaus of the sigmoid curve of the first reference signal 131. In contrast, the linear function of the second reference signal 132 has a unimodal distribution of the intensities as the second reference distribution 472. Due to such a transformation, the decision problem is reduced to establishing a unimodal or bimodal distribution in a corresponding measurement signal transformed into the distribution form.

FIG. 5A and FIG. 5B show schematic signal diagrams according to an exemplary embodiment. The schematic signal diagram in FIG. 5A is shown in the form of an intensity-time diagram or intensity-cycle diagram. In this case, the time t or a cycle number is plotted on the abscissa axis. An intensity I or signal intensity I is plotted on the ordinate axis. FIG. 5A shows the measurement signal 120, the first reference signal 131, and the second reference signal 132 from one of the above-mentioned figures in superimposed form. The schematic signal diagram in FIG. 5B is shown in the form of an intensity distribution-intensity diagram. In this case, the intensity I or signal intensity I is plotted on the abscissa axis. An intensity distribution P(I) is plotted on the ordinate axis. FIG. 5B shows the measurement signal distribution 175 from FIG. 1, the first reference distribution 471 from FIG. 4B, and the second reference distribution 472 from FIG. 4D, which are each obtained by transformation into a distribution form.

In other words, the measurement signal 120, the first reference signal 131, and the second reference signal 132 are transformed into intensity distributions. These distributions are now subjected to a cluster analysis with k=2. The bimodal and the unimodal distribution of the generic functions or reference distributions 471 and 472 robustly form two clusters, wherein the measurement signal distribution 175 or raw data distribution falls into one of the two clusters or categories, as a function of its modality.

FIG. 6A and FIG. 6B show schematic signal diagrams according to one exemplary embodiment. The schematic signal diagram in FIG. 6A is shown in the form of an intensity-time diagram or intensity-cycle diagram. In this case, the time t or a cycle number is plotted on the abscissa axis. An intensity I or signal intensity I is plotted on the ordinate axis. FIG. 6A shows the measurement signal 120 from one of the above-mentioned figures. The schematic signal diagram in FIG. 6B is shown in the form of a noise-time diagram. In this case, the time t or a cycle number is plotted on the abscissa axis. A noise η is plotted on the ordinate axis. FIG. 6B shows the noise data 155 from FIG. 1 or the ascertained noise of the measurement signal.

In other words, FIG. 6A and FIG. 6B show a further step for obtaining robust decisions. As shown in FIG. 4A, FIG. 4B, FIG. 4C, and FIG. 4D and also FIG. 5A and FIG. 5B, a measurement signal 120 or a qPCR curve in the distribution form or intensity distribution can be characterized with little effort as amplifying or non-amplifying. However, if the noise η is high, the distribution of the intensities is thus accordingly stretched and compressed. The moments of the distribution curves, for example expected values/peaks, width of the peaks/variances, and symmetry of the curve/skewness deviate from ideal curves and become less prominent, i.e., more demanding to characterize. The existing noise η is, however, a measurable variable, which may be ascertained from the raw data of the measurement signal 120. Since air bubbles in microfluidic devices or systems provide a large interference contribution, the noise η is not constant as a function of the cycle number or time t, but increases with greater cycle number or progressing time t. This is to be attributed to the number and size of air bubbles becoming greater over the reaction time of the analysis. Using a sliding window approach, the noise η is measured as a function of the cycle number or the time t using a suitable noise measure, for example, local standard deviation, local signal-to-noise ratio, or the like. The noise data 155 are obtained therefrom, which show the functional relationship of noise η and cycle number or time t for the present analysis.

FIG. 7A, FIG. 7B, and FIG. 7C show schematic signal diagrams according to one exemplary embodiment. The schematic signal diagrams in FIG. 7A and FIG. 7B are shown in the form of intensity-time diagrams or intensity-cycle diagrams. In this case, the time t or a cycle number is plotted in each case on the abscissa axes. An intensity I or signal intensity I is plotted in each case on the ordinate axes. FIG. 7A shows the first reference signal 131 and the second reference signal 132 from one of the above-described figures. FIG. 7B shows the adapted first reference signal 161 and the adapted second reference signal 162 from FIG. 1. The schematic signal diagram in FIG. 7C is shown in the form of an intensity distribution-intensity diagram. The intensity I or signal intensity I is plotted here on the abscissa axis. An intensity distribution P(I) is plotted on the ordinate axis. FIG. 7C shows the first reference distribution 171 and the second reference distribution 172 from FIG. 1, which are obtained by transforming the respective adapted reference signals 161 and 162 into a distribution form.

In other words, FIG. 7A, FIG. 7B, and FIG. 7C show how the noise graph or the noise data 155 from FIG. 6B are used to update the reference signals 131 and 132 using the same noise conditions to which the measurement signal is subjected. The reference signals 131 and 132 represent model data having ideal values, wherein a random number or pseudorandom number is added to each ideal value for each cycle of the analysis. This random number or pseudorandom number is generated from the noise interval of the signal measured in FIG. 6A or from the noise data from FIG. 6B. Two adapted reference signals 161 and 162 thus result, which have the same noise as the measurement signal. If the adapted reference signals 161 and 162 are transformed into the distribution form or intensity distribution, the ideal distributions are deformed in accordance with the measured noise, in particular compressed. The measurement signal and the matching reference distribution 171 or 172 are thus closer for the cluster analysis and the cluster decision is more robust.

FIG. 8 shows a flow chart of an evaluation process 800 according to one exemplary embodiment. The evaluation process 800 is executable in conjunction with the device from FIG. 1 or a similar device. A first block 810 of the evaluation process 800 represents the measurement signal. A following second block 820 represents a scaling of the measurement signal and a measurement of a cycle-dependent noise η(t). A following third block 830 represents an update of the first reference signal and the second reference signal using the noise η(t). An in turn following fourth block 840 represents a transformation of the measurement signal and also the updated reference signals into a distribution form. A following fifth block 850 represents a cluster analysis using the transformed measurement signal and the transformed updated reference signal by means of a k-means algorithm with k=2. A final sixth block 860 represents a decision according to the cluster analysis as to whether or not the measurement signal indicates an amplification.

The evaluation process 800 can also be referred to as a robust decision process. The evaluation process 800 is suitable in particular for evaluating qPCR having high noise. A qPCR curve in the form of the measurement signal is used as the input data of the evaluation process 800. The measurement signal is scaled in a first step and the cycle-dependent noise is measured as shown in the second block 820. Next, two reference signals or generic curves are generated or updated using the measured noise, as shown in the third block 830. The first reference signal has a qPCR-typical sigmoid curve and the second reference signal has a linear curve. The measurement signal, the first reference signal, and the second reference signal, which were processed in the above-described way, are then converted into an intensity distribution, as shown in the fourth block 840. Subsequently, the converted signals are classified by means of cluster analysis into two clusters, as shown in the fifth block 850. The measurement signal is then further treated like the reference signal, with which the measurement signal was associated during the cluster analysis. The evaluation process 800 can be or become integrated directly into evaluation software of a microfluidic device, such as the device from FIG. 1.

FIG. 9 shows a flow chart of a method 900 or analysis method 900 according to one exemplary embodiment. The method 900 is executable to perform an analysis of biological material. The method 900 is executable here in conjunction with the device from FIG. 1 or a similar device.

In a step 910 of reading, in the method 900 for analysis, a measurement signal, a first reference signal, and a second reference signal are read. The measurement signal represents acquired optofluidic data of the biological material. The first reference signal represents first optofluidic model data, which correspond to a first property of the biological material. The second reference signal represents second optofluidic model data, which correspond to a second property of the biological material.

Subsequently, in the method 900 for analysis, in a step 920 of ascertaining, a noise of the read measurement signal is ascertained to generate noise data. In turn following, in a step 930 of applying, the noise data is applied to the first reference signal and to the second reference signal to generate an adapted first reference signal and an adapted second reference signal. Subsequently, in a step 940 of transforming, the measurement signal, the adapted first reference signal, and the adapted second reference signal are transformed into a frequency distribution form to generate a measurement signal distribution, a first reference distribution, and a second reference distribution.

Subsequently, in the method 900 for analysis, in a step 950 of performing, a cluster analysis is performed using or on the measurement signal distribution, the first reference distribution, and the second reference distribution, to establish as a function of a result of the cluster analysis whether the biological material has the first property or the second property. Although it is not shown in the illustration of FIG. 9, the method 900 for analysis can also include a step of outputting or providing an analysis signal which represents a result of the analysis.

According to one exemplary embodiment, the method 900 for analysis includes a step 905 of acquiring the optofluidic data of the biological material to provide the measurement signal. The step 905 of acquiring is executable here before the step 910 of reading.

Optionally, the method 900 for analysis includes a step 915 of scaling the read measurement signal by projecting absolute values on a predefined value interval. The step 915 of scaling is executable here before the step 920 of ascertaining. In the step 920 of ascertaining, the noise of the scaled measurement signal is ascertained here.

If an exemplary embodiment includes an “and/or” linkage between a first feature and a second feature, this is to be read to mean that the exemplary embodiment includes both the first feature and also the second feature according to one embodiment and includes either only the first feature or only the second feature according to a further embodiment.

1. A method for analyzing biological material, the method comprising:
reading a measurement signal representing acquired optofluidic data of the biological material, a first reference signal representing first optofluidic model data corresponding to a first property of the biological material, and a second reference signal representing second optofluidic model data corresponding to a second property of the biological material;
ascertaining a noise of the measurement signal, to generate noise data;
applying the noise data to the first reference signal and to the second reference signal, to generate an adapted first reference signal and an adapted second reference signal;
transforming the measurement signal, the adapted first reference signal, and the adapted second reference signal into a frequency distribution form to generate a measurement signal distribution, a first reference distribution, and a second reference distribution; and
performing a cluster analysis using the measurement signal distribution, the first reference distribution, and the second reference distribution to establish as a function of a result of the cluster analysis whether the biological material has the first property or the second property.

2. The method as claimed in claim 1, further comprising:
acquiring the acquired optofluidic data of the biological material to provide the measurement signal.

3. The method as claimed in claim 1, wherein:
the measurement signal represents optofluidic data of the biological material acquired by a quantitative and/or qualitative polymerase chain reaction,
the first reference signal has a sigmoid curve,
the second reference signal has a linear curve,
in the reading of the first and second reference signals, the first property of the biological material results in an amplification of at least one target molecule of the biological material and the second property of the biological material results in an absence of the amplification of the at least one target molecule
the transforming of the measurement signal, the adapted first reference signal, and the adapted second reference signal includes transforming the adapted first and second reference signals in such a way that the first reference distribution is a bimodal distribution and the second reference distribution is a unimodal distribution.

4. The method as claimed in claim 1, wherein the performing of the cluster analysis includes using a k-means algorithm having a predefined distance measure, wherein the first reference distribution represents a first cluster, the second reference distribution represents a second cluster, and the result of the cluster analysis specifies whether, in consideration of the predefined distance measure, the measurement signal distribution falls into the first cluster or into the second cluster.

5. The method as claimed in claim 1, wherein, in the ascertaining of the noise, the noise of the measurement signal is ascertained over a curve of the analysis and/or via a sliding window process and/or using a noise measure to generate as the noise data a functional relationship between the noise and the curve of the analysis.

6. The method as claimed in claim 1, wherein the applying of the noise data includes adding random numbers or pseudorandom numbers dependent on the noise data to the first and second optofluidic model data of the first and second reference signals.

7. The method as claimed in claim 1, further comprising:
scaling the read measurement signal by projecting absolute values on a predefined value interval,
wherein the ascertaining of the noise includes ascertaining the noise of the scaled measurement signal.

8. A device for analyzing biological material, the device comprising:
a control unit configured to:
read a measurement signal representing acquired optofluidic data of the biological material, a first reference signal representing first optofluidic model data corresponding to a first property of the biological material, and a second reference signal representing second optofluidic model data corresponding to a second property of the biological material;
ascertain a noise of the measurement signal to generate noise data;
apply the noise data to the first reference signal and to the second reference signal to generate an adapted first reference signal and an adapted second reference signal;
transform the measurement signal, the adapted first reference signal, and the adapted second reference signal into a frequency distribution form to generate a measurement signal distribution, a first reference distribution, and a second reference distribution; and
perform a cluster analysis using the measurement signal distribution, the first reference distribution, and the second reference distribution to establish as a function of a result of the cluster analysis whether the biological material has the first property or the second property.

9. A computer program configured to execute and/or activate a control unit to:
read a measurement signal representing acquired optofluidic data of a biological material, a first reference signal representing first optofluidic model data corresponding to a first property of the biological material, and a second reference signal representing second optofluidic model data corresponding to a second property of the biological material;
ascertain a noise of the measurement signal to generate noise data;
apply the noise data to the first reference signal and to the second reference signal to generate an adapted first reference signal and an adapted second reference signal;
transform the measurement signal, the adapted first reference signal, and the adapted second reference signal into a frequency distribution form to generate a measurement signal distribution, a first reference distribution, and a second reference distribution; and
perform a cluster analysis using the measurement signal distribution, the first reference distribution, and the second reference distribution to establish as a function of a result of the cluster analysis whether the biological material has the first property or the second property.

10. A machine-readable storage medium, comprising:
at least one memory on which the computer program as claimed in claim 9 is stored.
